(19) United States
(12) Patent Application Publication
(10) Pub. No.: US 2012/0006715 A1
(43) Pub. Date: Jan. 12, 2012

(54) Feminine fresh all day

(76) Inventors: Joni Jane Bartels, Los Gatos, CA (US); Christian R. Bartels, Los Gatos, CA (US)

(21) Appl. No.: 12/803,960

(22) Filed: Jul. 12, 2010

(51) Int. Cl. B65D 85/84; A61P 31/00; A61K 36/00; A61K 36/752; A61K 36/53; A61K 36/534

(52) U.S. Cl. 206/524.1; 424/725; 424/747; 424/736

(57) ABSTRACT

To date no over-the-counter, non-toxic, self-use products like Feminine Fresh All Day have been offered to women who desire a quick external cleansing method for the feminine reproductive area after urination (refer to document “Patent Search”).

Currently, materials on the market include varieties of self-use wipes for external use that are either caustic, alcohol based, and/or other prescriptive internal applicators with associated chemical mixtures, solutions and concoctions to be used when infections, hormonal dysfunction, or age related dryness is present.

While these internal applications may have a higher value when those conditions are present, our invention purports that frequent external cleanings after urinating, using commonly known herbal and natural antibacterial phytochemical properties may help prevent the consequence of infections as a product of residual buildup of external bacteria after urination.

Additionally, a pleasant residual fragrance from this product can be well appreciated with use.

### BACKGROUND

For years I, Joni Jane Bartels, have noticed as a 52 year old female the recurrent potential for problematic changes in my reproductive anatomy concerning periodic mild to moderate infections.

While many medical doctors have recommended prescriptive lotions or internal medications as solutions for this common female related problem, I found the best answer over time in the obvious support through cleanliness.

And as I became more determined to meet knowledge about diet relationships and consistent patterns with proper cleaning of my anatomy along with natural changes in my hormone levels, I finally consulted with my now husband Dr. Christian Bartels.

From the compiling of his years of education and clinical experience and my accumulated information, we aspired together to develop a simple, natural and portable answer to this common female issue of health.

In 2010 we researched, developed and produced the first chemical template of test our product design. A liquid mixture composed of low concentrations of therapeutic herbal extracts and water.

I put my own body to use in the practical application and found a wonderfully simple method for use in travel situations that could be very easily carried. As well I found the likelihood of low grade infections and odors to be completely abated with the regular use of this new concoction showing that simple wiping may not be as effective as this combined with a mild regularly used therapeutic application.

### INTENDED USAGE

Women as busy, stressed out, office workers or homemakers can use this safe, easily applied system to control external bacterial buildup.

The plastic applicator bottle can be squeezed to express a small amount of therapeutic liquid in spray form onto the external vaginal area before wiping dry. This lends to cleanliness and improvement of post urination odorous tendencies during busy daily life.

### Formula

93.75 ml mixture (3 ounces)

Active Ingredients

0.05 ml Extract of Lavender (included in “Lavender”) with 4.95 ml 5×Whole Leaf Aloe Vera Juice Conc.

0.05 ml Extract of Peppermint (included in “Peppermint”) with 4.95 ml 5×Whole Leaf Aloe Vera Juice Conc. 0.05 ml Tea

Tree Oil (included in “Tea Tree”) with 4.95 ml 5×Whole Leaf Aloe Vera Juice Conc.

0.05 ml Extract of Grapefruit (included in “Grapefruit”) with 4.95 ml 5×Whole Leaf Aloe Vera juice Conc.

0.05 ml each extract (included in “Ultimate”) with 5×Whole Leaf Aloe Vera Juice to reduced 4.80 ml

Inactive Ingredients:

88.75 ml of purified water

What is claimed is:

1. Aqueous mixture that when shaken and sprayed becomes an emulsion which can be used as an externally applied cleaning and disinfecting agent to human genitalia after urination.

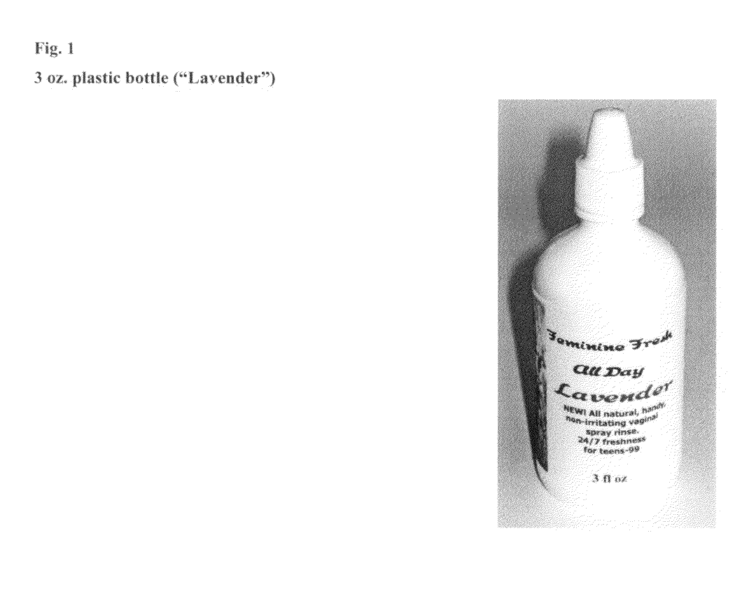

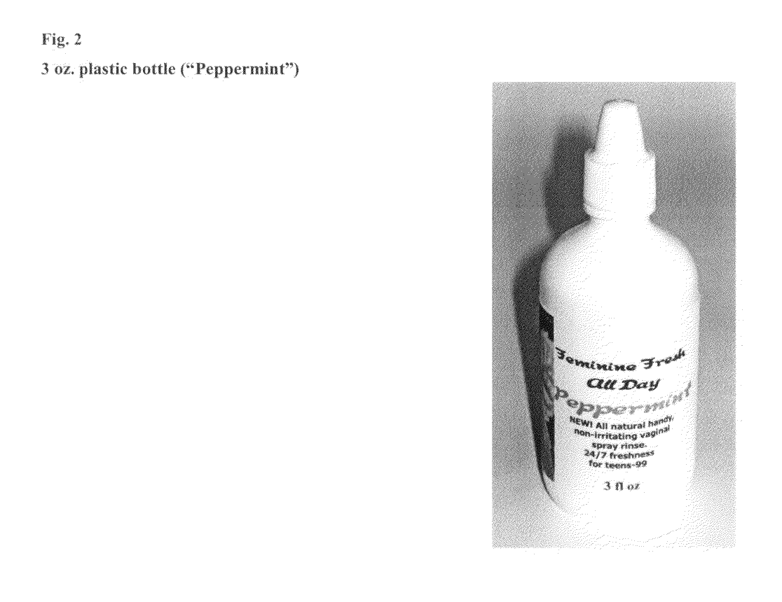

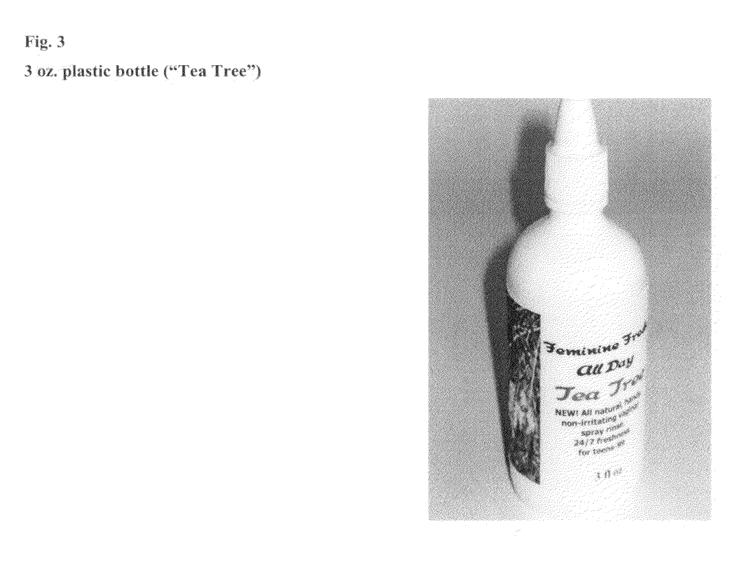

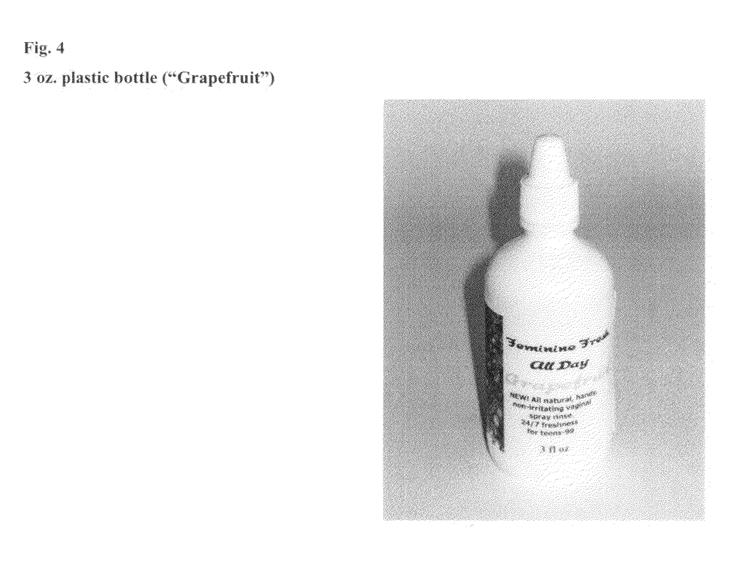

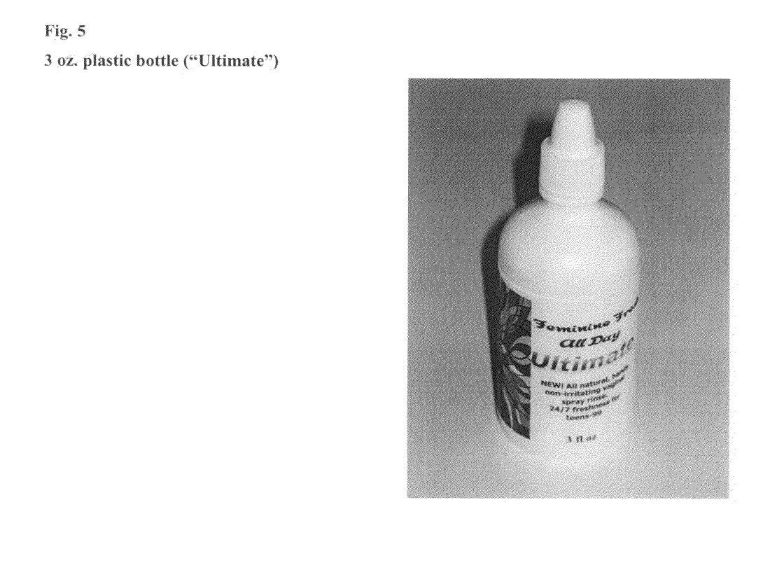

2. Herbal constituents, individually or combined, in mixture have therapeutic value:
a. Lavender oil (FIG. 1—“Lavender”) in low concentration as solutes in purified water.
b. Peppermint oil (FIG. 2—“Peppermint”) in low concentration as solutes in purified water.
c. Tea Tree oil (FIG. 3—“Tea Tree”) in low concentration as solutes in purified water.
d. Grapefruit oil (FIG. 4—“Grapefruit”) in low concentration as solutes in purified water.
e. Combined items a. b. c. and d. (FIG. 5—“Ultimate”) in low concentration as solutes in purified water.

3. Plastic 1.5 oz. and 3.0 oz. bottles used for storage and consumer usage.
